(19) United States
(12) Patent Application Publication
(10) Pub. No.: US 2007/0255108 A1
(43) Pub. Date: Nov. 1, 2007

(54) WATER RESISTANT L.E.D. POCKET OTOSCOPE

(76) Inventor: James David Schmitz, Midland, TX (US)

Correspondence Address: James Schmitz, 2819 Maxwell Dr, Midland, TX, 79705, US

(21) Appl. No.: 11/381,026

(22) Filed: May 1, 2006

(51) Int. Cl. A61B 1/06

(52) U.S. Cl. 600/175

(57) ABSTRACT

The otoscope including an elongated body member having a water resistant substantially hollow interior, a head member attached to the distal end of the body member, a power source positioned in the water resistant substantially hollow interior of the body member, a light emitting diode positioned in the water resistant substantially hollow interior portion of the body member and being in selective electrical communication with the power source, and a light emitting diode holder positioned within the water resistant substantially hollow interior portion of the body member and having a concave surface facing the head member, the light emitting diode holder having an aperture formed through a central axis thereof, the aperture being sized to receive the distal end of the light emitting diode. There is a seal between the end of light emitting diode that extends through the aperture and the aperture itself thus exposing only the end of the LED light to the external environment. Since the Light emitting diode burns cool unlike incandescent light sources there is no harm to it from moisture even after it has been burning for extended periods. The otoscope head member also contains a light reflecting surface positioned in the path of light being emitted from the end of the light emitting diode that extends out the aperture, the reflecting member being positioned at an angle to direct the light at a 90 degree angle down the otoscope speculum and into the ear canal for use in visualization via the magnifying lens.

### BACKGROUND OF THE INVENTION

1. Field of the Invention

Embodiments of the invention generally relate to an improved small light weight otoscope having a low current draw light emitting diode as a source of illumination incased in a water resistant housing.

2. Description of the Related Art

Otoscopes are generally used by physicians, nurses, and health-givers to examine ear canals and ear drums. Conventional hand-held otoscopes with incandescent light sources have been developed to illuminate ear canals and magnifying lenses to magnify the illuminated area for examination. However, difficulties have been encountered with the useful lifetime of the light bulbs and batteries used in conventional devices, as the current draw of conventional bulbs generally runs the battery of a conventional otoscope dead in a matter of hours. Additionally, conventional bulbs used in conventional otoscopes have been shown to be easily damaged from both accidental dropping of the otoscope, as well as from the jarring and movement associated with normal operation and transportation of conventional otoscopes. Further, the light provided by conventional otoscope bulbs, if brighter, would provide a much improved view for the practitioner, which generally results in improved diagnosis and treatment. There is also a need for a durable light weight small pocket otoscope with low energy consumption that can be used in the field during inclement or rainy conditions. An otoscope that can also be easily transported from place to place on a person with minimal concern of bulb damage due to the fragile filament breaking as is now found on all pocket otoscopes using incandescent light sources. The otoscope can also be made very small and portable since it can make use of small batteries since it has very low power consumption.

Therefore, in view of the reliability, longevity, and cost challenges presented by conventional otoscopes, there is a need for a new an improved small light weight pocket otoscope that uses a low-power light source that provides improved battery lifetime, while also providing a light source that is not easily damaged or rendered inoperable during normal operation and transportation of the device. A device that will not be harmed by using it during rainy or wet conditions while working outdoors or in the field since the light emitting diode burns cool and it's light emitting surface is not effected by moisture.

### SUMMARY OF THE INVENTION

The present invention generally relates to an improved otoscope. Embodiments of the invention generally provide a hand-held light emitting diode (LED) pocket otoscope for assisting physicians and caregivers in viewing and evaluating the condition of the ear canal and eardrum. The LED pocket otoscope includes a battery source electrically connected to an LED light source, a mirror for redirecting a beam of light from the LED light source through an opening that allows the redirected light to enter into a patient's ear canal, and a lens positioned to magnify the user's view of the illuminated ear canal. The light source, power source and electrical elements are enclosed in a water resistant housing.

Embodiments of the invention may further provide a hand-held otoscope for examining areas of low visibility, the hand-held otoscope including a light emitting diode source for emitting a light beam, a battery source electrically connected to the light emitting diode, and a reflecting surface fixed and oriented with respect to the light emitting diode to reflect the light beam from its original direction to another direction. The hand-held otoscope may further include the light emitting diode source having a useful lifetime of at least 100,000 hours, and further, the light emitting diode source may require less than about three volts and thirty milli-amperes during operation.

Embodiments of the invention may further provide a hand-held otoscope for examining the ear, the hand-held otoscope including a water resistant tubular housing and a top, the tubular housing including a light emitting diode source for emitting a light beam, a battery source electrically connected to the light emitting diode, and a water resistant removable cap enabling the battery source to be inserted and removed. The top may include a front and back end, a deflecting surface for deflecting the direction of the light beam along a path through the front end, and a magnifying lens attached to the back end and permitting a user to view a magnified image of an object illuminated by the deflected light beam. The front end may further include a seating region, the seating region being sized and proportioned for receipt of a speculum that may be attached to the seating region. The back end may include a screw ridging, the magnifying lens including a mating screw ridging for removably attaching to the back end. Additionally, the otoscope of the invention may include including a water resistant pressure switch the selectively connects the battery source and light emitting diode, where the pressure switch may operate to activate a circuit between the battery source and light emitting diode when depressed by a user.

Embodiments of the invention may further provide a pocket otoscope having an LED light source wherein the beam of light emitted from the LED light source is directed along a cylinder cavity and deflected by a mirror at a 90 degree angle through an aperture. The pocket otoscope includes a magnifying lens that is located at the top of the cylinder cavity and oriented to provide a view through the aperture at the object illuminated by the light beam. Two AAA batteries are loaded into the rear of the cylinder cavity to provide the power for the LED.

Embodiments of the invention may further provide an otoscope. The otoscope may include an elongated body member having a water resistant substantially hollow interior, a head member attached to a first distal end of the body member, a power source positioned in the water resistant substantially hollow interior portion, a light emitting diode positioned in the water resistant substantially hollow interior portion and being in selective electrical communication with the power source, and a light emitting diode holder positioned within the water resistant substantially hollow interior portion and having a concave surface facing the head member, the light emitting diode holder having an aperture formed through a central axis thereof, the aperture being sized to receive the light emitting diode there through and a light reflecting member positioned in the otoscope head, the reflecting member being positioned to direct the light at a 90 degree angle down the speculum of the otoscope.

Embodiments of the invention may further provide an LED otoscope. The LED otoscope may include an elongated body member having a water resistant substantially hollow interior, a head member attached to a first distal end of the body member, a power source, comprising two triple A size batteries, positioned in the water resistant substantially hollow interior portion proximate a second distal end of the body member, and a light emitting diode positioned in the water resistant substantially hollow interior portion and being in selective electrical communication with the power source. The otoscope may further include a light emitting diode holder positioned within the water resistant substantially hollow interior portion and having a reflective concave surface facing the head member, the light emitting diode holder having an aperture formed through a central axis thereof, the sealed aperture being sized to receive the light emitting diode there through, and a light reflecting member positioned in a path of light being emitted from the light emitting aperture, the reflecting member being positioned at an angle to direct the light at a 90 degree angle down the speculum of the otoscope.

Embodiments of the invention may further provide a LED otoscope. The LED otoscope includes a circular elongated body member having a water resistant substantially hollow interior, a head member attached to the distal end of the body member, the head member having a magnifying lens and a speculum positioned thereon as well as containing a reflective member, a power source, comprising two triple A size batteries, positioned in the water resistant substantially hollow interior portion of the otoscope body, and a light emitting diode positioned in the water resistant substantially hollow interior portion and being in selective electrical communication with the batteries. The otoscope further includes a light emitting diode holder positioned within the water resistant substantially hollow interior portion and having a reflective concave surface facing the head member, the light emitting diode holder having an aperture formed through a central axis thereof, the aperture being sized to and sealed around the end of the light emitting diode that extends there through. An otoscope head that contains a light reflecting member positioned in a path of light being emitted from the light emitting aperture, the reflecting member being positioned at an angle to direct the light at a 90 degree angle down a speculum of the otoscope, the reflecting member having a reflective coating thereon that comprises a base protective material having a thickness of about 50 microns, a reflective material having a thickness of about 65 microns, and an outer transparent protective layer having a thickness of about 50 microns.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a transparent side view of an embodiment of the LED pocket otoscope of the invention;

FIG. 2 illustrates a transparent forward view of an embodiment of the LED pocket otoscope of the invention;

FIG. 3 illustrates a more detailed sectional view of a central portion of an exemplary otoscope of the invention.

### DETAILED DESCRIPTION

The present invention generally provides an improved otoscope that utilizes a low power, durable, water resistant, LED light source.

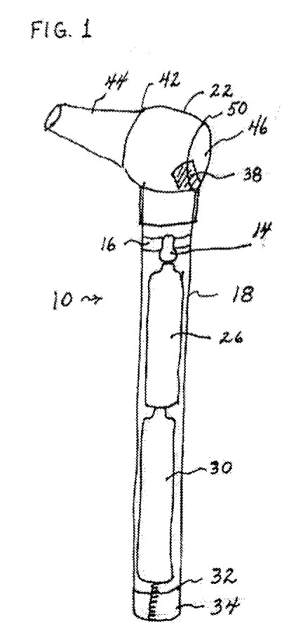

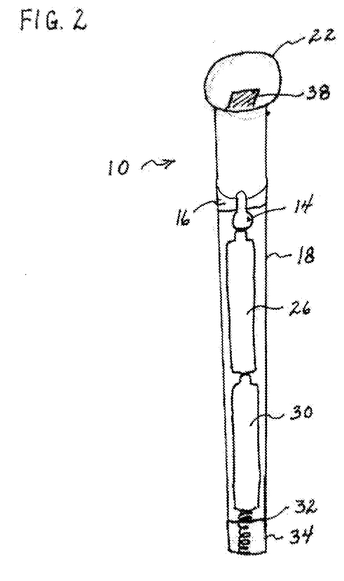

Referring to FIG. 1 and 2 collectively, an exemplary embodiment of a pocket otoscope 10 of the invention is shown. The pocket otoscope generally utilizes an intense, full spectrum, light emitting diode (LED) 14 that has at least an average bulb life of 100,000 hours of continuous use at low power to illuminate hidden areas and is useable by physicians and caregivers for examining ear canals and eardrums. In the exemplary embodiment, pocket otoscope 10 generally includes a tube-shaped frame 18 and head portion 22. The tube-shaped frame 18 may enclose a source of electrical power for the LED 14, which may be two removable 1.5 V AAA sized batteries 26, 30, which are insertable through rear end 32 of frame 18 and connected to power LED 14. The LED 14 is generally removably seated in a holder 16. The tube-shaped frame 18 may have a circular, square, octagonal, or other external cross-sectional surface that allows the exterior surface to be easily gripped and oriented by the user. The tube-shaped frame 18, for example, may be formed with a light weight metal, such as aluminum or stainless steel, or alternatively, from a rigid plastic material. A switch 34 is generally twistably or rotatably secured to the frame 18 and is configured to electrically connect batteries 26, 30 in series with LED 14. LED 14 generally draws less than about 30 milli-amperes of electrical current when illuminated or active, which allows the otoscope of the invention to have an improved service life per battery change over conventionally illuminated otoscopes.

During operation, light emitted from LED 14 is directed along frame 18 into a head portion 22. Head portion 22 generally includes a neck that slides over and attaches to frame 18. In one embodiment, the head 22 is attached by glue to the top of frame 18; while in another embodiment, the head 22 may be removable and adjustably positioned on frame 18 according to a preference of a user using other securing means that are known in the art. The head 22 may be formed with a rigid black colored plastic, and generally includes light reflecting surface 38, such as a mirror or polished metal, mounted and oriented at an angle that is calculated to deflects the light emitted from the LED 14 through an opening at front end 42 of head 22. In one embodiment of the invention, the reflecting surface may be positioned at a 90 degree angle. It may be appreciated that depending upon the orientation of front end 42 to the direction of the beam from LED 14, the angle of deflecting surface 38 may be adjusted to direct the beam through the aperture of front end 42. Front end 42 generally includes a connector base for detachably seating speculum 44 that incorporates a conventional mating connector, such as a screw grooved inner circumference for mating with a screw grooved outer connector surface of speculum 44. A variety of specula 44 may be attached and detached to front end 42 depending upon the needs of the user.

Head 22 also includes magnifying lens 46, such as 2.5× power magnification optical lens, which is located at rear end 50 and oriented to provide a magnified view through the aperture of the object illuminated by the light beam through speculum 44. Head 22 is generally placed on the frame 18 in a way that the light coming from LED 14 is focused on to reflective surface 38 and out through cylindrical speculum 44 at a 90 degree angle. In use, a speculum 44 that is generally removably attached to the otoscope of the invention, is inserted into an ear canal for examination allowing the reflected light to pass through speculum 44 and enter the ear canal. The magnifying lens 46 is located at the opposite end of head 22 (rear end 50) such that the inside of the ear canal may be viewed with the use of the intense light provided by LED 14 and reflected into the ear canal by deflecting surface 38.

In another embodiment of the otoscope of the invention, rear end 50 of head 22 has a screw ridged inner surface about its circumference to accommodate a magnifying lens with mating screw ridged surface to be screwed or twisted into position on head 22. In this embodiment, various powers of magnifying lens may be utilized with the otoscope depending upon the need or preference of a user or caregiver.

In yet another embodiment of the otoscope of the invention, switch 34, as described above, does not close the circuit connecting batteries 26, 30 and LED 14. In this embodiment, a pressure pad is incorporated along frame 18 that includes an electrical connection completing the circuit connecting batteries 26, 30 and LED 14 when the pressure pad is depressed. The pressure pad may be formed with a resilient rubber that is deformable when pressed and then regains shape when pressure is released. Alternatively, there may be a spring mechanism that pushes the pressure pad outward when not depressed. When the pressure pad is not depressed the circuit is open and LED 14 and batteries 26, 30 are not active.

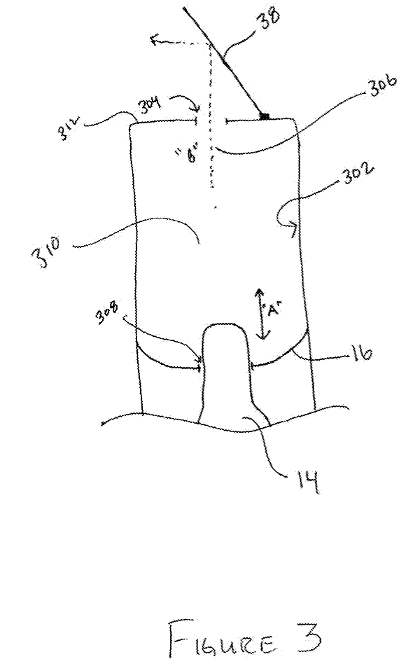

FIG. 3 illustrates a more detailed sectional view of a central portion of an exemplary otoscope of the invention. The central portion, for example, generally includes the top of the batteries, the LED, and the reflector. The LED holder 16 is shown as having a concave surface that has an aperture 308 formed in a central portion thereof. The aperture 308 is sized to receive an upper most portion of the LED 14 there through. The holder 16 may generally be covered with a reflective material or coating, such as a coating similar to the reflective coating on reflective surface 38. Additionally, the inner walls 302 of the tube 18 that are above the LED 14 may also be coated with a reflective material, such as a coating similar to the coating covering reflective surface 38. Thus, the combination of the holder 16 and the inner walls 302 having a reflective material thereon provide for a more intensified light being generated by the otoscope of the invention, and the light continually reflects in the light chamber 310 before exiting the chamber 310 through an aperture 304 formed in an upper body closing member 312. The aperture 304 is positioned to direct light exiting from the light chamber 310 toward the reflective surface 38, as shown by dotted line “B”.

In another embodiment of the invention, the LED of the invention may comprise a variable wavelength LED. The LED may be powered by different voltages or currents, which cause the LED to emit different wavelengths of light. The otoscope of the invention may be equipped with a user activated switch that operates to adjust the wavelength of light being emitted from the LED to suit the particular application, i.e., a different wavelength of light may assist with diagnosis of specific conditions.

In yet another embodiment of the invention, the LED 14 may be vertically movable. More particularly, LED 14 may be actuated by the user in the direction of arrow “A” illustrated in FIG. 3. The movement of the LED 14 may allow for adjustment of the light intensity of the otoscope, as when the LED is lowed in the holder 16, less light is emitted into the light chamber 310. The adjustment of the LED may be tied to a screw type on/off switch of the otoscope where the body 18 is twisted relative to the head of the otoscope to turn the LED on, and further, additional twisting would increase the LED's entrance into the light chamber 310, and thus increase the light intensity.

The reflective surface 38 may generally include a semi rigid backing member that has a reflective material thereon. The reflective material may be an ultra-high reflectivity, mirror-like optical enhancement film. For example, the film may be a multi-layer, non-metallic polymer that operates as a high performance reflector. The reflective material's high reflectivity generally remains relatively constant across the visible spectrum, thereby eliminating any unwanted color shifts. The reflective material also provides a very low coefficient of thermal expansion, which provides for minimal linear expansion of the film under heat and cooling cycles, which provides excellent thermal stability. The reflective material may include a base protective layer having a thickness of about 50 microns, a reflective layer having a thickness of about 65 microns, and an outer transparent protective layer having a thickness of about 50 microns. The total thickness of the reflective material is less than about 165 microns, for example.

The foregoing has outlined, in general, the physical aspects of the invention. In reference to such, there is to be a clear understanding that the present invention is not limited to the method or detail of construction, fabrication, material, or application of use described and illustrated herein. Any other variation of fabrication, use, or application should be considered apparent as an alternative embodiment of the present invention. It will also be understood that, in addition to examining ear canals and their anatomy, the device can be used to exam, magnify, and light up any object that would lend itself in need of these said qualities for examination. One example of another use might be to locate a splinter while using tweezers to remove the splinter. Another example might be a locksmith wanting to visualize the tumblers on a lock or a collector wanting to magnify and visualize details of coins or stamps.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

While the foregoing is directed to embodiments of the present invention, other and further embodiments of the invention may be devised without departing from the basic scope thereof, and the scope thereof is determined by the claims that follow.

1. An otoscope, comprising:
an elongated body member having a water resistant substantially hollow interior;
a head member attached to a first distal end of the body member;
a power source positioned in the water resistant substantially hollow interior portion proximate a second distal end of the body member;
a light emitting diode positioned in the water resistant substantially hollow interior portion and being in selective electrical communication with the batteries;
a light emitting diode holder positioned within the water resistant substantially hollow interior portion and having a concave surface facing the head member
the light emitting diode holder having an aperture formed through a central axis thereof
the aperture being sized to receive the distal end of the light emitting diode there is a seal between the end of light emitting diode that extends through the aperture and the aperture itself thus exposing only the end of the LED light to the external environment
a head member attached to a first distal end of the body member, the head member having a magnifying lens and a speculum positioned thereon
a head member that also contains a light reflecting member being positioned at an angle to direct the light at a 90 degree angle down the otoscope speculum and into the ear canal,
a reflecting member having a reflective coating thereon that comprises a base protective material having a thickness of about 50 microns, a reflective material having a thickness of about 65 microns, and an outer transparent protective layer having a thickness of about 50 microns.

2. The otoscope of claim 1, further comprising a portion of the water resistant substantially hollow interior portion being coated with a light reflecting coating.

3. The otoscope of claim 2, wherein the LED is vertically movable relative to the diode holder.

4. The otoscope of claim 1, wherein the LED has a service life of at least about 100,000 hours.

5. The otoscope of claim 4, wherein the LED has a current draw of less than about thirty milliamps.

6. The otoscope of claim 5, wherein the LED has a voltage of about 3 volts.

7. The otoscope of claim 1, wherein the power source is connected to the LED through a twisting action of the elongated body member relative to the head member.

8. The otoscope of claim 1, wherein the angle of the light reflecting member is about 45 degrees from vertical.

9. The otoscope of claim 1, wherein the reflecting member has a reflecting coating thereon, the reflecting coating comprising a multilayer, non-metallic, optical enhancement film.

10. The otoscope of claim 9, wherein the optical enhancement film comprises a high reflectivity that remains constant across a visible spectrum of light.

11. The otoscope of claim 9, wherein the optical enhancement film comprises a base protective material having a thickness of about 50 microns, a reflective material having a thickness of about 65 microns, and an outer transparent protective layer having a thickness of about 50 microns.

12. An LED otoscope, comprising:
an elongated body member having a water resistant substantially hollow interior;
a head member attached to a first distal end of the body member;
a power source, comprising two triple A size batteries, positioned in the water resistant substantially hollow interior portion proximate a second distal end of the body member;
an light emitting diode positioned in the water resistant substantially hollow interior portion and being in selective electrical communication with the power source; The aperture being sized to receive the distal end of the light emitting diode that is also sealed around the end of the light emitting diode and the aperture leaving only the end of the light emitting diode exposed to the exterior environment,
a light reflecting member positioned in a path of light being emitted from the light emitting aperture, the reflecting member being positioned at an angle to direct the light toward the ear canal down the speculum of the otoscope.

13. The light emitting diode of claim 12, wherein the reflecting member has a reflecting coating thereon, the reflecting coating comprising a multilayer, non-metallic, optical enhancement film.

14. The otoscope of claim 13, wherein the optical enhancement film comprises a high reflectivity that remains constant across a visible spectrum of light.

15. The otoscope of claim 14 wherein the optical enhancement film comprises a base protective material having a thickness of about 50 microns, a reflective material having a thickness of about 65 microns, and an outer transparent protective layer having a thickness of about 50 microns.

16. The otoscope of claim 15, wherein the LED has a service life of at least about 100,000 hours.

17. The otoscope of claim 16, wherein the LED has a current draw of less than about thirty milliamps.

18. The otoscope of claim 17, wherein the LED has a voltage of about 3 volts.

19. An LED otoscope, comprising:
a circular elongated body member having a water resistant substantially hollow interior;
a power source, comprising two triple A size batteries, positioned in the water resistant substantially hollow interior portion;
a light emitting diode positioned in the water resistant substantially hollow interior portion and being in selective electrical communication with the batteries;
a light emitting diode holder positioned within the water resistant substantially hollow interior portion and having a concave surface facing the head member
the light emitting diode holder having an aperture formed through a central axis thereof
the aperture being sized to receive the distal end of the light emitting diode there is a seal between the end of light emitting diode that extends through the aperture and the aperture itself thus exposing only the end of the LED light to the external environment
a head member attached to a first distal end of the body member, the head member having a magnifying lens and a speculum positioned thereon
a head member that also contains a light reflecting member being positioned to direct the light emitted from the light emitting diode at a 90 degree angle down the otoscope speculum and into the ear canal.
a reflecting member having a reflective coating thereon that comprises a base protective material having a thickness of about 50 microns, a reflective material having a thickness of about 65 microns, and an outer transparent protective layer having a thickness of about 50 microns.
